(19) United States
(12) Patent Application Publication
(10) Pub. No.: US 2012/0264623 A2
(43) Pub. Date: Oct. 18, 2012

(54) SYSTEM AND METHOD FOR THE CLONAL CULTURE OF EPITHELIAL CELLS AND APPLICATIONS THEREOF

(75) Inventors: Nicolas Fortunel, Saint-Yon (FR); Michele Martin, Chateaufort (FR); Pierre Vaigot, Ablons Sur Seine (FR)

(73) Assignee: COMMISSARIAT A L'ENERGIE ATOMIQUE ET AUX ENERGIES ALTERNATIVES, Paris (FR)

(21) Appl. No.: 12/918,028

(22) PCT Filed: Feb. 18, 2009
(86) PCT No.: PCTEP2009051912

§ 371 Date: Aug. 17, 2010

(30) Foreign Application Priority Data
Feb. 19, 2008 (FR) 0851054

(51) Int. Cl. C40B 30/00; C12Q 1/68; C12Q 1/06; C40B 40/02; C12N 5/071; C12Q 1/04

(52) U.S. Cl. 506/7; 435/395; 435/34; 435/39; 506/14; 435/6.11; 506/9

(57) ABSTRACT

The invention relates to means and methods for evaluating and using the specific properties of a particular epithelial cell present in a biological sample. Accordingly, the invention relates to a system for the culture of epithelial cells, in which at least one clonal culture is sown with a single epithelial cell directly extracted from a biological sample of epithelial tissue. The invention also relates to a method for the culture of epithelial cells, that particularly comprises the production of clonal cultures, each being sown with a distinct and unique epithelial cell directly extracted from a biological sample of epithelial tissue, the evaluation of the cellular growth in the clonal cultures, and advantageously the analysis of the capacity of the cellular material from the clonal cultures to reconstruct a three-dimensional epithelium representative of native tissue. The invention is adapted for the parallel implementation of a very large number of clonal cultures, in particular for making large-scale tests.

The present invention relates to the field of cell biology and of tissue engineering.

More specifically, the invention proposes means and methods with which the specific properties of a particular epithelial cell present in a biological sample may be evaluated and utilized.

Thus, the object of the present invention is a system for cultivating epithelial cells, in which at least one clonal culture is sown with a single epithelial cell directly extracted from a biological sample of epithelial tissue.

The invention further relates to a method for cultivating epithelial cells, comprising at least the steps of:

a) extracting one or more epithelial cells directly from a biological sample of epithelial tissue;

b) optionally, selecting at least one population and/or subpopulation of epithelial cells from the cells extracted in step a);

c) producing a clonal culture sown with a distinct and single epithelial cell directly stemming from step a) or b); and

d) qualitatively and/or quantitatively evaluating cell growth in the clonal culture of step c).

Further, the invention is directed to applications of such a system or method.

In vitro systems and methods dealing with epithelial tissues such as the epidermis, find applications in fields as diverse as medical research and clinical development, tissue engineering and toxicology.

The different pluristratified epithelial tissues (notably the epidermis, the cornea, mucous tissues . . . ) share a certain number of common features which impose constraints for designing large scale in vitro test architectures. These general characteristics are well exemplified in the case of the epidermis.

The epidermis is the most superficial structure of the skin and notably ensures the barrier function thereof. In majority consisting of keratinocytes, it is renewed on average every 28 days. This tissue comprises 4 layers which correspond to the 4 steps of the differentiation program which the keratinocytes undergo during their migration from the basal layer, the deepest layer, towards the stratum corneum, the most superficial layer. The continuous physiological process of renewal of the various layers of keratinocytes is called keratinopoiesis.

The basal layer of the epidermis, which includes only one monocellular layer, is the germinative compartment. It is at this layer that proliferation of the keratinocytes is carried out. Among basal keratinocytes, a small proportion of cells called stem cells is found, for which it is recognized that they are at the origin of the long term renewal of the epidermis. The immediate offspring of the stem cells is called a population of progenitors. The latter ensure rapid short term renewal of the epidermis.

The stem cell notion, within the human inter-follicular epidermis, therefore defines the compartment located the most upstream in the hierarchy of keratinopoiesis. These cells are notably characterized by significant self-renewal capacity, which progenitor cells do not have, and a fortiori the keratinocytes engaged in differentiation. Further, an important property of the stem cells is to durably preserve the potential for regenerating and rebuilding the epidermal tissue.

Like the other pluristratified epithelial tissues, the epidermis therefore consists of a heterogeneous assembly of cells having variable differentiation (or immaturity) degrees. It is generally recognized that basal keratinocytes represent about 10% of the whole of the keratinocytes of the epidermis, and the compartment of the epidermal stem cells only of the order of 0.1%.

Taking into account the quantitative needs required for applying the present methods for functional evaluation and screening, the cell material routinely collected from skin biopsies, i.e. the whole of the keratinocytes obtained after dissociation of an epidermis sample, allows the building-up of cell banks with a sufficient size for use at an industrial scale. However, the material used for building up this type of banks corresponds to a heterogeneous assembly of cells, comprising basal keratinocytes having different growth capacities and supra-basal keratinocytes in the course of differentiation and no longer having any growth capacity.

As regards banks of keratinocytes, for example intended for industrial production of kits of rebuilt epidermises, a standard method consists of freezing the cells at the end of a single multiplication step in a culture, so as to form a stock of multiple equivalent ampoules, which are kept in liquid nitrogen. Depending on the needs, the cell ampoules are thawed out and placed in culture in order to achieve a second multiplication step. At the end of the two successive multiplication steps, the keratinocytes have generally carried out of the order of 10 doublings of population. This type of approach was moreover used for analyzing the heterogeneity of the growth potential of human keratinocytes (Barrandon and Green, 1987). The authors first produced primary cultures derived from the epidermis, which they froze in liquid nitrogen. Sub-confluent secondary cultures were prepared from frozen primary cultures. The clones obtained after cloning (third cultivation step or “third pass”) were classified into three categories: holoclones (rapid growth), paraclones (limited growth) and meroclones (intermediate population).

The keratinocytes conventionally obtained after two successive multiplication steps may be used for producing models of rebuilt tissues. Applying these systems as they are to rare cell material, such as stem cells, is on the other hand impossible at an industrial scale where vast test campaigns have to be conducted.

All in all, several types of models compatible with the conducting of large scale in vitro test campaigns are presently available. The biological material used in these tests may be: 1) immortalized cell lines; 2) banks of normal cell extracted from tissue biopsies and amplified in culture; 3) rebuilt three-dimensional tissues. However, for applying such models at a large scale, it is necessary to have available large amounts of cell material. For example, in the case of tests conducted on normal cells, in vitro amplified cell populations are used, which modifies certain properties thereof depending on the applied culture parameters. Further, if the focus is on sub-populations of rare cells, such as progenitor cells or epidermal stem cells, these cells are obtained in insufficient amounts from tissue biopsies.

Gangatirkar et al. (2007) describe a method with which an epidermis may be rebuilt from total keratinocytes or from sub-populations sorted on the basis of distinct phenotypes. Both proposed options consist of using cells directly after extraction from the tissue and after cell sorting on the one hand, and using the cell material after an expansion phase in culture on the other hand. However, the quantitative needs of cell material for applying the described method remain unsuitable for conducting large scale test campaigns in parallel. Further, the cell material used corresponds to a complex mixture of different cells, the capacity of which for rebuilding an epidermis is used in a global way.

The heterogeneity of the cells composing the pluristratified epithelial tissues, and notably that of the keratinocytes of the epidermis, is therefore a limiting factor in the elaboration of in vitro test strategies.

Indeed, insofar that the cells have certain characteristics which are specific to them, they are capable of responding differently to a stimulus or a stress. The same applies for pathological epithelial tissues. For example, carcinomas are very heterogeneous tumors, in which a small proportion of tumoral stem cells represent a key target for the treatments.

Further, when cells having distinct characteristics are mixed in a culture, the specific behavior of some of them may be modified or ignored within the mixture. An “averaged” result is thus observed over the whole of the cultivated cells. Thus, the structural and functional properties of an epidermis rebuilt from a heterogeneous global cell population (see for example Larderet et al., 2006) are the result of the whole of the properties of the cells put in presence of each other. A thereby rebuilt epidermis can therefore by no means reflect the specific properties of a single cell.

Further, the applied culture conditions may more or less severely modify the intrinsic characteristics of the cells. Indeed, it is well known that the fact of placing cells from an epithelial tissue in an artificial culture environment leads to modification of their native characteristics. Consequently, epithelial cells used after one or more culture steps have cell material which is no longer comparable with cells directly stemming from a tissue sample. These modifications in particular relate to the specific phenotype of the studied cells. For example, it has been shown that the cultivation of human keratinocytes freshly isolated from an epidermis perturbs the expression of adhesion molecules and markers used for defining a phenotype of stem cells, and this, in a variable way depending on the culture medium used (Lorenz et al. 2008).

Eventually, standard solutions which consist of working from heterogeneous complex cell populations are not adequate for meeting present medical, clinical and industrial needs.

There is therefore a need for means and methods which allow access to the individual properties of the cells stemming from pluristratified epithelial tissues, while being suitable for the application of large scale in vitro test campaigns, even in the case of a cell material 100 to 1,000 times rarer than the general populations which are presently used.

The present invention for the first time meets this need by proposing culture means and methods which (i), because of their clonal nature, allow access to the individual and specific properties of cells directly stemming from pluristratified epithelial tissues, (ii) preserve the individual potential of said cells, (iii) even when they are applied at a large scale, do not consume much cell material, which makes them suitable for the study and exploitation at an industrial scale of the less represented cells (stem and progenitor cells), and (iv) allow cell growth levels to be reached which are much greater than those obtained with known tools.

Thus, an object of the present invention relates to a clonal culture system of epithelial cells optimized for evaluating and exploiting the specific properties of a single cell, in which a culture support comprises at least one clonal culture sown with a single epithelial cell directly extracted from a biological sample of epithelial tissue.

Advantageously, said culture support comprises at least two parallel clonal cultures, each of said cultures being sown with a distinct and unique epithelial cell directly extracted from said biological sample.

Preferably, such a system appears as a biochip. The clonal cultures sown in parallel are then for example microcultures. In practice, the biochip may notably be made from culture plates comprising multiple distinct wells, for example 6, 24, 96 wells or more. Alternatively, the biochip may have as a support, a glass plate or a plate in any other suitable material, on which multiple microsurfaces are created, intended to receive the cells, for example by a surface treatment allowing the cells to adhere and to grow thereon. Biochips made on plates may be physically divided into compartments, for example by means of grids, or chemically, for example following a surface treatment of the plates which prevents the cloned cells from migrating out of their respective culture microsurfaces.

Another object of the present invention relates to a method for the clonal cultivation of epithelial cells, optimized for evaluating and exploiting properties specific to a single cell, comprising at least the steps of:

a) extracting one or more epithelial cells directly from a biological sample of epithelial tissue;

b) optionally, selecting at least one population and/or sub-population of epithelial cells from the cells extracted in step a);

c) producing a clonal culture sown with a distinct and single epithelial cell directly stemming from step a) or b); and

d) qualitatively and/or quantitatively evaluating cell growth in the clonal culture of step c).

Interestingly, the cells used in the method, object of the present invention, may be total populations of cells directly extracted from these tissues, and/or sub-populations thereof, sorted on the basis of specific characters. Thus, cell material stemming from step b) may advantageously correspond to one or more sub-populations enriched in epithelial progenitors and/or stem cells.

During step c) (which may be considered as a primary growth step), the thereby extracted cell preparation is used for initiating parallel clonal cultures or microcultures. The question is of sowing the cells of interest individually under conditions allowing their growth, for example in separate culture wells. In practice, clonal sowings may be carried out in an automated way with technologies such as notably flow cytometry or microfluidics. Preferably, step c) comprises the production of at least two parallel clonal cultures, each of said cultures being sown with a distinct and single epithelial cell directly stemming from step a) or b).

In particular, during step d), the growth of the cloned cells is analyzed on the basis of one or more quantitative and/or qualitative parameters such as:

the frequency of obtained clones relatively to the number of sown cultures: clone-forming efficiency [CFE];

the proliferative potential of the clones: number of cells making up each clone at a given culture time;

the phenotype of clones: differentiation degree of the cells making up the clones, expression of molecular markers.

Thus, against every expectation, the inventors were able to observe that the growth potential of cells cultivated according to the clonal culture method of the present invention is higher than that of cells cultivated according to standard procedures (see Example B hereafter).

In a particular embodiment, the clonal culture method of the invention comprises at least the steps of:

a) extracting one or more epithelial cells in the form of monocellular suspension(s), directly from a biological sample of epithelial tissue;

b) selecting at least one population and/or sub-population of epithelial cells from the cells extracted in step a);

c) producing a clonal culture sown with a distinct and single epithelial cell stemming from step b); and

d) qualitatively and/or quantitatively evaluating cell growth in the clonal culture of step c).

According to another embodiment, the method, object of the present invention, further comprises step e) consisting of amplifying the cell population of the clonal culture of step c), or its offspring, by one or more successive sub-cultures.

The question here is to produce from cell clones obtained at the end of the primary growth step c), long term parallel independent cell cultures via successive sub-cultures amplified for several weeks. Depending on the needs, the amplification may be conducted over periods for example ranging from 2 to about 8 weeks, or even longer (cf. Exemplary embodiment No. 1 C.2, below). With these independent cultures, it is possible to obtain a large amount of cells which may be frozen and stored in the form of one or more banks of cells, for subsequent use.

Clonal cell banks which may thereby be obtained also represent an object of the present invention. These banks are distinguished from existing banks by the fact that they integrate clonal cell cultures which give them highly specific structural and functional properties.

In another additional or alternative embodiment, the method according to the invention further comprises step f) consisting of evaluating the tissue reconstruction potential of the cell population of the clonal culture of step c) or of its offspring. More specifically, step f) preferably consists of using the cell population of the clonal culture of step c), or its offspring, in order to rebuild a three-dimensional tissue, so as to evaluate its tissue reconstruction potential.

In practice, for carrying out this step, cells from primary clonal cultures or microcultures may be detached from their culture support, and they may then be used individually for each clone of interest, in order to produce a three-dimensional organotypic culture model (for example, a rebuilt epithelium, epidermis or skin).

Three-dimensional tissues rebuilt from clonal cultures, which may be obtained at the end of step f) of the method according to the invention, are part of the objects of the present invention. These tissues are produced according to a novel three-dimensional organotypic model since the structural and functional characteristics of the tissues, object of the invention, are quite specific insofar that they result from the properties of a single cell. Such tissues are notably selected from various epithelial tissues, the skin, the epidermis.

Further, biochips comprising at least one tissue, as described above, form another object of the invention. These biochips may for example be formed from microcultures made within a three-dimensional gel made from a biomaterial compatible with cell growth. Systems based on multiple rebuilt three-dimensional microtissues may also be contemplated, each being generated independently, directly within the biochip, without any prior culture step.

In another additional or alternative embodiment, the method according to the invention further comprises step g) consisting of evaluating the long term expansion potential of the cell population of the clonal culture of step c). More specifically, step g) preferably consists of sub-cultivating the cell population of the clonal culture of step c), under conditions promoting cell expansion until exhaustion of the expansion potential, so as to evaluate the long term expansion potential of said cell population.

For example, cells stemming from primary clonal cultures or microcultures may be detached from their culture support, and then be sub-cultivated under conditions promoting their multiplication, until exhaustion of their multiplication potential.

In another additional or alternative embodiment, the method according to the invention further comprises step h) consisting of evaluating the clone-forming potential of the offspring of the cell population of the clonal culture of step c). More specifically, step h) preferably consists of evaluating the clone-forming potential of the offspring of the cell population of the clonal culture of step c), by means of a quantitative test of clonogenicity in which strictly clonal secondary cultures and/or low density cultures allowing growth of individualized colonies are made.

For this, for example, the cells forming each clone may be detached from their culture support and a quantitative clonogenicity test may be conducted for each of them. Thus, strictly clonal secondary cultures and/or low density cultures allowing the growth of individualized colonies may for example be produced.

Essentially, within the scope of the present invention, the initial biological sample is a sample of healthy or diseased epithelial tissue, for example obtained by biopsy in a mammal, preferably in humans. In particular, the tissue sample may be selected from epithelia, for example the interfollicular epidermis of adult or neonatal human skin, the cornea, mucosas, hair follicles. Samples of diseased epithelial tissues are for example obtained by biopsy of patients affected with a genetic disease (such as xeroderma pigmentosum, bullous epidermolyses, etc.), by biopsy of cicatricial skin (notably in badly burnt persons). The diseased epithelial tissues may also be tumoral tissues (carcinomas, etc.).

The biological sample may possibly comprise cells from epithelial (notably keratinopoietic) differentiation of pluripotent stem cells selected from embryonic, fetal and induced pluripotent stem cells. As examples, mention will be made of cells having epithelial potential, stemming from fetal stem cells: cells from the ectodermal embryonic layer, cells from epithelial tissues, keratinocytes, etc. The cells having epithelial potential stemming from so-called “induced” pluripotent stem (IPS) cells are generated by reprogramming cells stemming from adult tissue which may be differentiated cells.

Advantageously, the epithelial cells directly extracted from the biological sample are single healthy or diseased cells selected from progenitor cells, stem cells, keratinocytes.

Another object of the present invention relates to a clonal culture system (or to a kit comprising such a system) obtained by applying at least steps a) to c) of the method according to the invention. These systems have particular properties inherent to the fact that they derive from clonal cultures. The kits may for example be diagnostic kits, tests for evaluating biological activity, toxicity tests, etc. It is quite clear for one skilled in the art that the terms of “test”, “kit” and possibly “system” may be equivalent here depending on the context in which they are used.

In a particular embodiment, a clonal culture system for epithelial cells optimized for evaluating and exploiting specific properties of a single cell, comprises, within the context of the invention, a culture support in which at least one clonal culture is sown with a single epithelial cell directly extracted from a biological sample of epithelial tissue according to the steps a) to c) of the method described earlier.

The present invention also relates to applications of the method and to uses of the various means (system, kit, cell bank, tissue, biochip) described above.

Preferred examples of such uses and applications are:

for identifying and selecting agents having a biological activity of interest. An “agent” may be a candidate molecule which is tested for its biological activity and which is selected depending on the applications, said activity may be positive (for example, for selecting effectors of pharmaceutical, therapeutic, cosmetic interest, etc.) or negative (for example, for selecting toxic molecules). Alternatively, an “agent” may be of a non-chemical nature, for example UV rays, visible light, ionizing radiations, magnetic waves, etc.;

for treatment and/or diagnosis (notably in vitro) and/or prognosis (notably in vitro) in the field of cell and/or gene therapy, notably in the field of grafts;

for studying the behavior and/or the structural and/or functional individual properties specific to a single cell, a cell sub-type, a cell sub-population, or a cell population;

for producing one or more tools of functional genomics, which are notably useful for inducing phenomena of gain or loss in biological activity, for medical purposes and/or in any type of functional exploration. For example, mention will be made of interfering RNAs, over-expression and/or repression, viral or non-viral vectors, etc.;

for evaluating the efficiency of agents having biological activity such as molecules of pharmaceutical or cosmetic interest, and/or evaluating the efficiency of treatments with such agents (molecules or other types of stimuli, for example waves, light, radiations, physical parameters, etc.).

Eventually, the various objects of the present invention as compared with the presently available means and methods have the following considerable advantages:

(i) The possibility of producing parallel microculture series, initiated from a single cell, which in practice allows the application of large scale test campaigns targeting rare sub-populations, poorly represented within tissues, which cannot be contemplated in standard models which are great consumers of cell material.

(ii) The possibility of initiating clonal cultures from individually sown cells of a selected phenotype, for example in microwells, immediately after being selected from the tissue, which gives the possibility of contemplating the setting into place of test strategies on cell material which has not undergone a multiplication step in culture, and therefore is less likely to have been modified by artificial culture parameters before conducting the tests, in particular when the treatment or stimulus is immediately applied after sowing microcultures.

(iii) The fact of having access for the first time to the behavior of cells individually placed in culture, which provides the possibility of describing and quantifying the biological properties and the specific responses of the different cells forming a population of interest, allowing analysis of cell heterogeneity, which is not possible in standard models which use cell populations globally.

Thus, the various objects of the present invention prove to be useful in very many fields. In addition to the examples of applications already described above, mention may notably be made in a non-limiting way of:

(i) Evaluation of the efficiency of agents having a biological activity: function gains.

Evaluation of the efficiency of compounds bearing a beneficial biological activity (for example, molecules of pharmaceutical interest, cosmetic actives):

Conducting screening campaigns allowing quantification of the effects of biological actives at the scale of a single isolated cell: action of a treatment on the actual target cell, impact on its offspring.

Possibility of developing strategies of evaluation tests targeted on poorly represented populations, such as normal or pathological stem cells.
Examples:

test of effectors capable 1) of inducing multiplication of stem or progenitor cells of pluristratified epithelial tissues, 2) promoting maintenance of the stem nature in culture in the offspring of isolated stem cells;

test of novel anti-cancer molecules on stem cells isolated from carcinomas.

(ii) Problems of toxicology: function losses, cancer transformation.

Estimation of the impact of stress and toxic agents at the scale of treated cells in isolation, after selection on the basis of specific criteria. These tests may selectively be applied to the cells responsible for long term renewal (stem cells) and short term renewal (progenitors) of epithelial tissues.
They also allow depending on the type of targeted cells, the design of tests adapted to prognosing and distinguishing acute or belated deleterious effects on a tissue or organ:

Conducting toxicological tests allowing estimation of the innocuousness of a treatment or, on the other hand, quantification of its toxic effects: short term effect on the actual isolated cell, consequences on its offspring.

Evaluating the impact of genotoxic aggressions: at the scale of cells studied in isolation, acquisitions of damages to DNA, transmission of mutations and/or genetic abnormalities to the offspring, consequences on organogenesis, etc.

(iii) Technology of biochips: parallel/massively parallel models of quantification and qualification of biological responses.

Screens of functional genomics on two-dimensional cell cultures and/or three-dimensional organotypic models.

High throughput screening of molecules bearing a biological activity/detection of deleterious properties (“high throughput screening” [HTS]).

(iv) Cell and gene therapy.

Qualification of samples of cells intended to be grafted, and/or intended to be used for producing grafts of rebuilt tissues:

Tests in culture allowing an estimation of the regenerative potential of cells intended for clinical use: growth potential individually estimated on cells under a clonal condition.

Prognosis tests of the capacity of engraftment of tissues rebuilt in vitro: estimation of maintenance or loss of growth potential of cells used for producing grafts.
Validation of gene transfer protocols in a clinical perspective:

Evaluation of the efficiency of a genetic correction protocol: estimation of the frequency of cells actually corrected at the end of the gene transfer method.

Evaluation of the stability of genetic correction: transmission to the offspring of individualized cells.

(v) Cell and tissue engineering.

Cell systems and organotypic models compatible with the application of tests on cells followed in isolation and their offspring:

Parallel production of cell banks, each formed by the offspring of a single cell placed in culture and in isolation.

Models of normal or pathological tissues rebuilt in vitro generated from the offspring of a single cell placed in culture under a clonal condition.

The following figures illustrate, in connection with examples below, embodiments of the present invention:

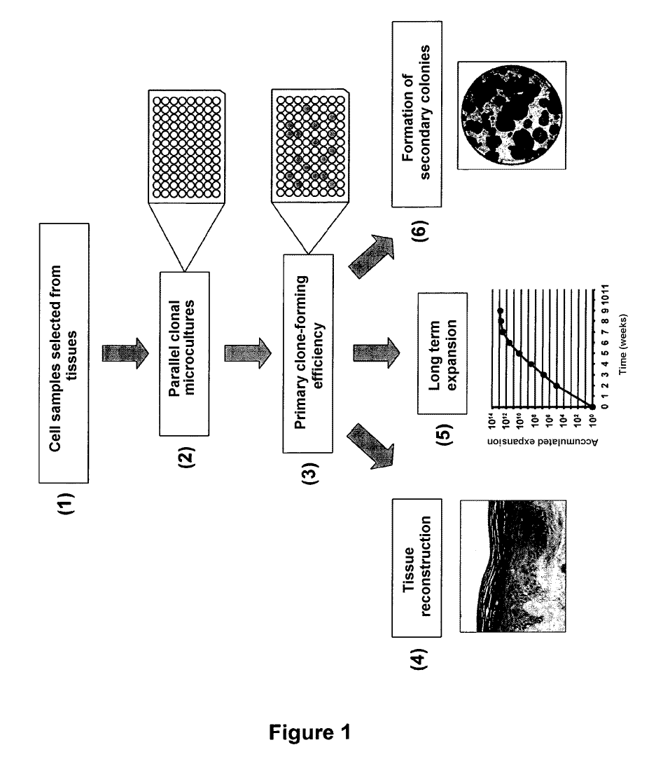

FIG. 1: a diagram illustrating an embodiment of the method according to the invention;

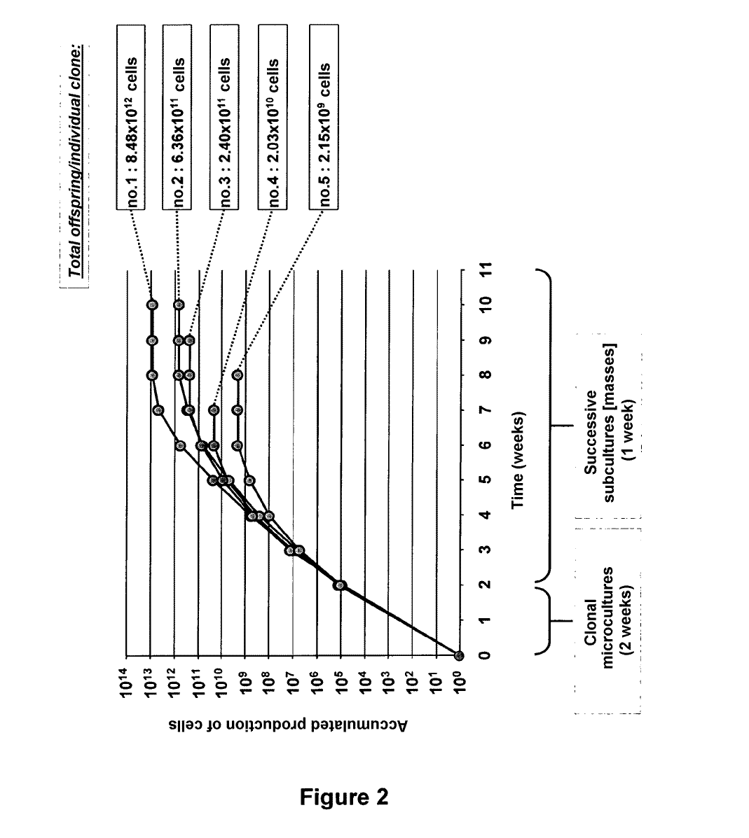

FIG. 2: a graphic illustration of the result of a long term expansion experiment for producing banks of multiple keratinocytes, each stemming from the offspring of a single cell;

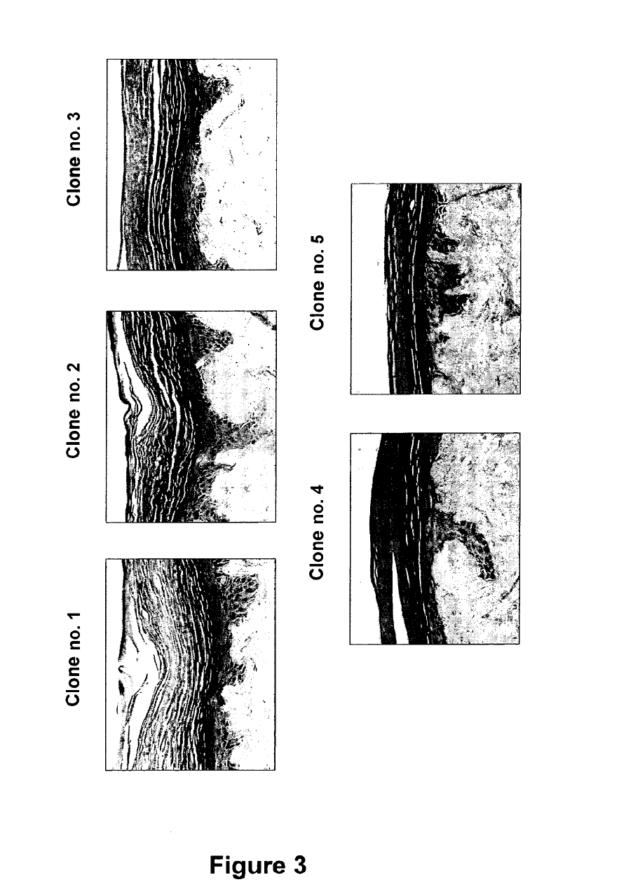

FIG. 3: results of an experiment for producing multiple rebuilt epidermises, each stemming from the offspring of a single cell;

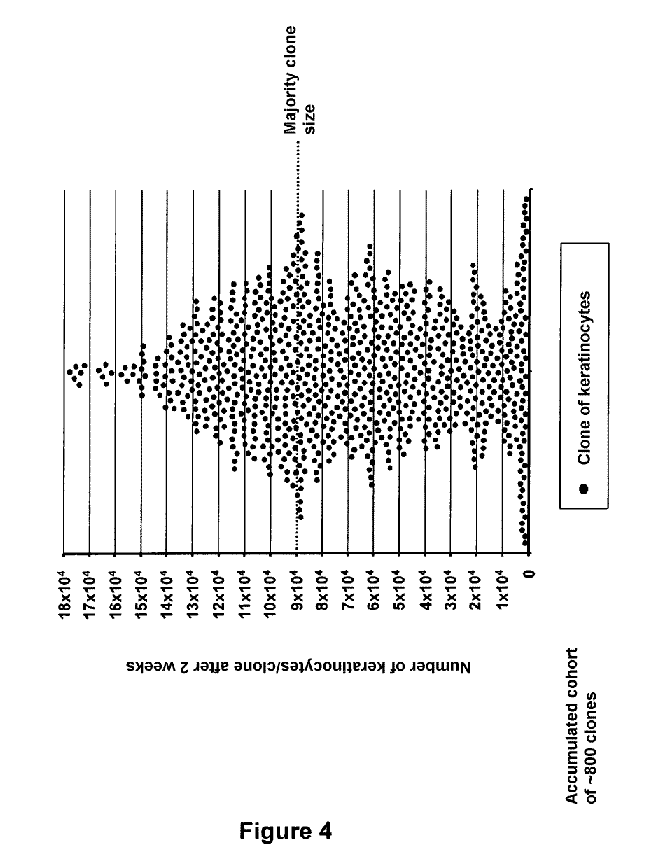

FIG. 4: results of the evaluation of short term clonal growth of basal keratinocytes Itg∝6^{strong} placed in culture individually;

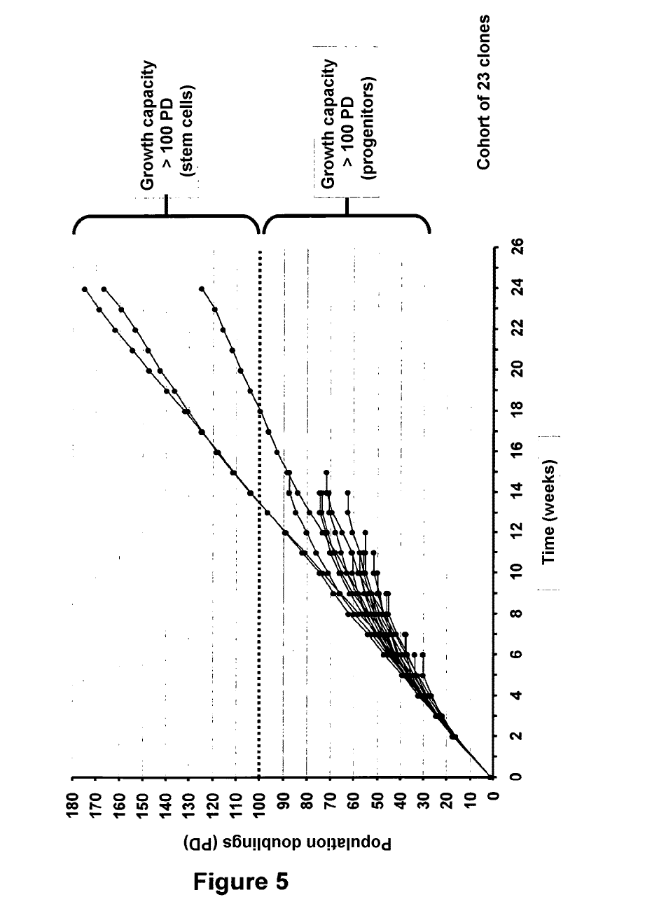

FIG. 5: results of an experiment in which long term cultures initiated from basal keratinocytes Itg∝6^{strong} individually placed in culture are quantified;

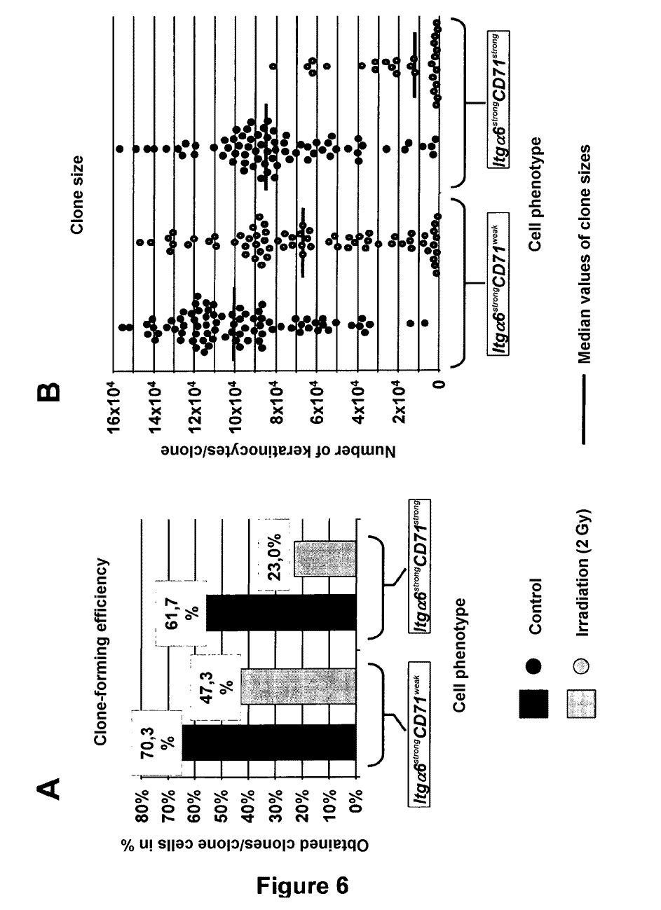

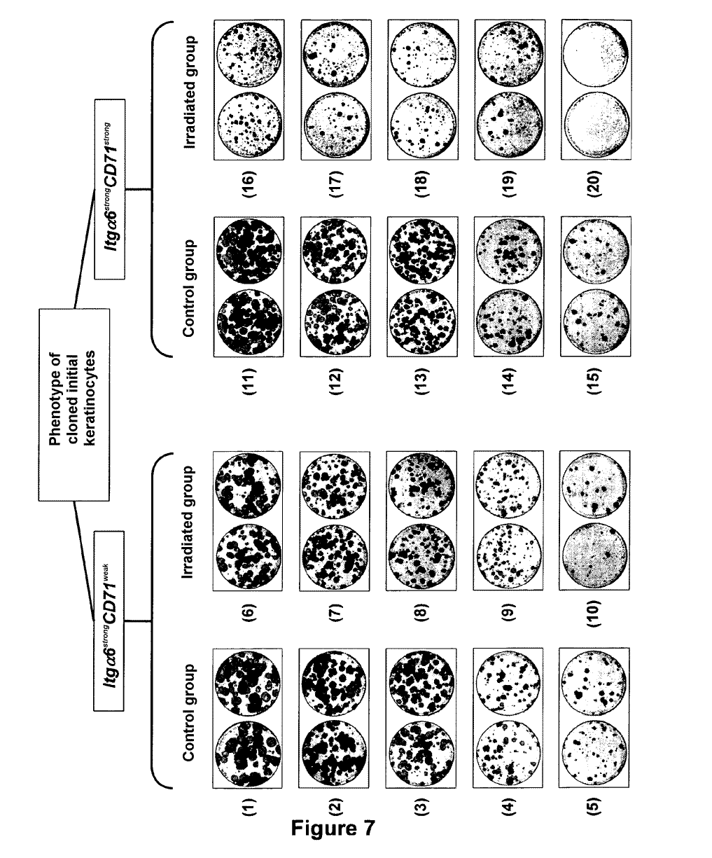

FIG. 6: a graphic illustration of the results of an experiment where the impact of irradiation on epidermal keratinocytes of distinct phenotypes was quantified at the scale of a single isolated cell;

FIG. 7: results of an experiment in which the functional test of parallel clonal microcultures was used for evaluating the consequences of irradiation carried out on an isolated cell, on the growth potential of its offspring, and in which the behavior of epidermal keratinocytes of distinct phenotypes was compared;

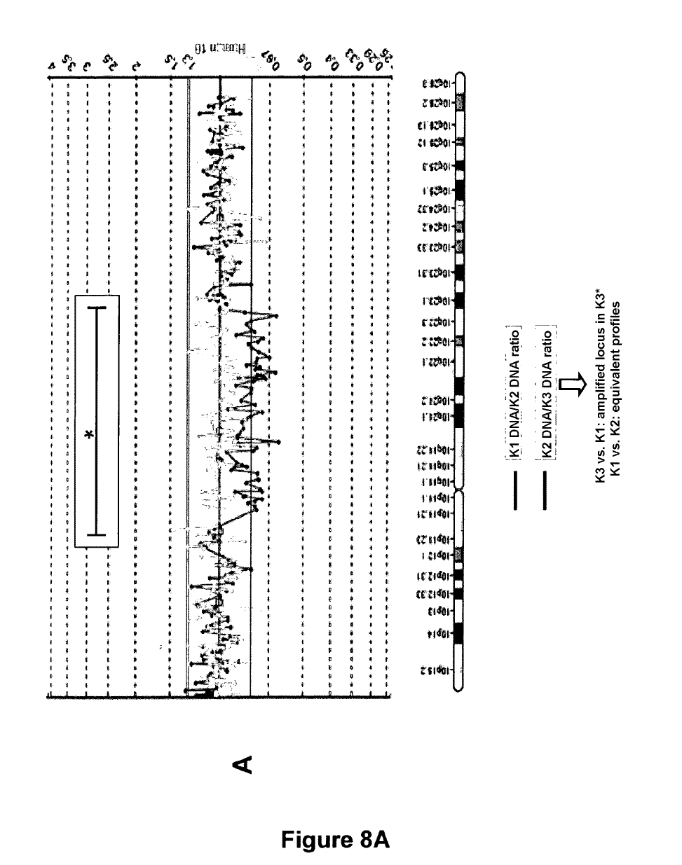

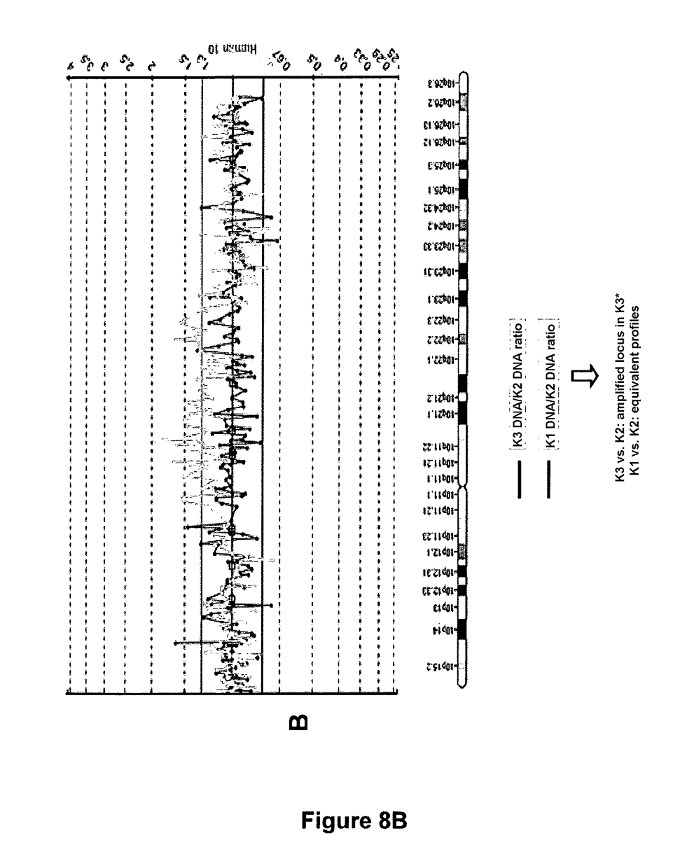

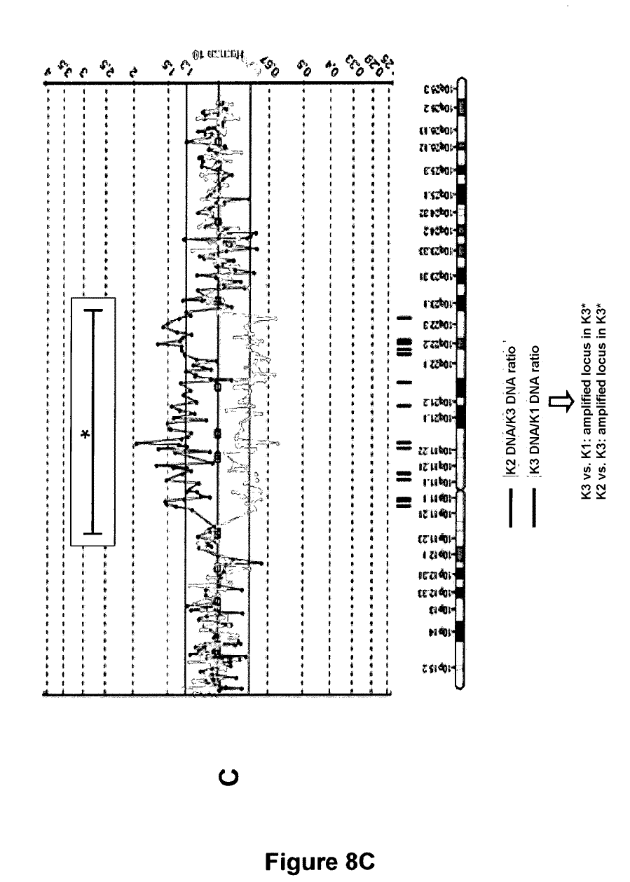

FIGS. 8A, 8B, 8C: result of a search for abnormalities at the chromosome 10 by CGH chips:

Offspring of two non-irradiated keratinocytes: K1 and K2
Offspring of an irradiated keratinocyte: K3.
Particular embodiments and advantages of the present invention are described in the exemplary embodiments below, which deal with keratinocytes from adult human interfollicular epidermis.

These examples are provided purely as an illustration; they do not limit by any means the object and scope of the invention.

### EXAMPLES

### A—Experimental Procedures

A-1—Preparation of the Cell Material

A-1-1—Epithelial Cells Intended to be Used for Clonal Cultures

The tissue biopsies which in the example described here are biopsies of adult human skin, were first of all decontaminated, for example by soaking them in a physiological solution containing betadine. In order to allow separation between the epithelial tissue and the associated connective tissue (in the present case, the epidermis and dermis), the samples were then incubated in an enzyme solution at 4° C. for 10-15 hours (Gibco trypsin). At the end of this enzymatic digestion step, the tissue samples were dissected with fine tweezers, so as to isolate the epithelial portion of the tissue (here, the interfollicular epidermis). The enzymatic treatment completed by a mechanical dissociation step by suctions and discharge with a pipette, allows extraction of the keratinocytes which make up the fragments of epithelia. The cell suspension was finally filtered on a sieve with a mesh of 50-70 microns (BD Falcon), in order to remove the cell aggregates. At the end of these steps, the cell samples appear as monocellular suspensions, which may be used for sowing clonal cultures.

A-1-2—Fibroblasts Used as Supporting Cells

In the described examples, epithelial cells (keratinocytes obtained from interfollicular epidermis) were studied under a clonal condition in a culture system using as support a nutritive layer of fibroblasts made unable to multiply by gamma irradiation with a dose of 60 Grays. Thus, these cells remained static but live, and they supported the growth of the studied epithelial cells. These fibroblasts may notably be extracted from the dermal portion of skin biopsies. To do this, dermis fragments were incubated in an enzymatic solution consisting of a mixture of dispase (Roche) and of collagenase (Roche) for 2-4 hours at 37° C. Digestion by the enzymes, combined with mechanical stirring, allows extraction of the fibroblasts. After removing non-digested tissue fragments by filtration on a sieve (BD Falcon), and then by washing, the obtained fibroblasts were then placed in culture in a medium consisting of 90% DMEM (Gibco) and 10% serum of bovine origin (Gibco), in order to be amplified. After multiplication in culture, the fibroblasts were irradiated, and then frozen so as to be stored until use.

A-2—Immuno-Phenotypic Labellings

Epithelial cells used for illustrating certain embodiments of the invention are keratinocytes having a strong expression level of α6 integrin (Itgα6 or CD49f) and a weak expression level of the receptor of transferrin (CD71): phenotype Itgα6^{strong} CD71^{weak}. For achieving the labeling with fluorescent antibodies required for defining this phenotype, the cell samples were placed in suspension in physiological saline buffer (PBS) supplemented with 2% bovine albumin serum (SAB) (Sigma), and then incubated for 10 minutes at 4° C. with mouse immunoglobulins (Jackson Immuno-Reasearch), in order to saturate the non-specific binding sites of the antibodies. Labelling of the antigens CD49f and CD71 was then carried out by adding specific antibodies coupled with fluorochromes, and then by incubation for 30 minutes at 4° C.: anti-CD49f-PE (phycoerythrin) (clone GoH3, BD Pharmingen) and anti-CD71-APC (allophycocyanin) (clone M-A712, BD Pharmingen). After washing the antibodies in excess, the samples were ready to be used for sowing clonal cultures.

A-3—Automated Sowing of Clonal Culture Microcultures

A-3-1—Sowing

In the described exemplary embodiments, the clonal microcultures of keratinocytes were sown in an automated way with a flow cytometer equipped with a cloning module (MoFlo, Cytomation). Excitation of the fluorochromes coupled with the labelling antibodies was carried out by using a 488 nm argon laser (Coherent) and a 630 nm laser diode. The signals emitted by phycoerythrin (PE) and allophycocyanin (APC) were respectively detected and quantified in wavelength windows of 580±30 nm and 670±30 nm. The sorting criterion selected in the present case corresponded to keratinocytes having the phenotype Itgα6^{strong} CD71^{weak} and accounting for about 1% of the total keratinocytes: a sub-population of keratinocytes described as being enriched in epidermal stem cells (Li et al., 1998).

A-3-2—Quality Control of the Clonal Sowings

In parallel with the clonal cultures intended to be used for the tests and studies (culture conditions described hereafter), series of clonal sowings were carried out with the purpose of validating the procedure for depositing the cells. These depositions were carried out under strictly identical technical conditions, but in a medium more favourable for locating the cells than the one used for their growth. This observation medium may for example be a physiological saline buffer (PBS) added with Hoechst 33342 (Sigma) with a concentration of 10 micrograms/ml. The Hoechst is a DNA coloring agent which is fluorescent under UV-excitation. Under these conditions, the individually sown cells in a large number of culture wells may be located, which allows verification of the efficiency of the method. Each microculture should contain a single cell, never two and the frequency of empty wells should be minimized.

A-4—Culture Conditions

A-4-1—Primary Clonal Growth

In the described examples, microcultures of keratinocytes were carried out in culture plates comprising 96 wells in which collagen of type I was adsorbed (Biocoat, Becton-Dickinson). The day before the sowing of epithelial cells under a clonal condition, a nutritive layer of irradiated fibroblasts was set into place in the culture wells. These supporting cells were sown at a density of 6,000 cells/cm². The culture medium used for growing the keratinocytes was based on a mixture of DMEM (Gibco) medium and of Ham F12 (Gibco) medium, added with serum of bovine origin (Hyclone). This basic medium was notably supplemented with EGF (Chemicon), with insulin (Sigma), with hydrocortisone (Sigma), with adenine (Sigma), with triiodothyronine (Sigma), with L-glutamine (Gibco), and with a solution of antibiotics and antimycotics (Gibco). After automated sowing of the keratinocytes in an amount of one single cell per well, the cultures were maintained in culture at 37° C. in an atmosphere comprising 90% humidity, in the presence of 5% CO₂, in the present case for 2 weeks. At the selected time, counting of the wells in which a cell clone had developed was carried out, and the number of keratinocytes forming each clone was then determined individually, after detaching the cells by trypsination (Gibco).

A-4-2—Study of the Tissue Reconstruction Potential

In the described examples, the tissue reconstruction potential of the offspring of keratinocytes initially placed in culture individually was demonstrated in an epidermal reconstruction model on de-epidermized dead human dermis (Regnier et al., 1986). For preparing dermal supports, human skin samples were incubated for 10 days at 37° C. in PBS buffer, in order to detach the epidermis from them, which was then removed. The epidermis-free dermal samples were cut into squares of about 1 cm². They were then subject to several successive freezing/thawing cycles which led to the killing of the dermal cells. The obtained a cellular dermises were stored at −20° C. until use. The process for rebuilding a three-dimensional epidermis comprised 2 successive culture steps. The cell samples from clonal microcultures were first of all sown on the dermal supports and cultivated for 1 week in immersion in a comparable culture medium similar to the one used for the primary clonal culture (composition example described above). The second step of the epidermal reconstruction method consisted in placing the epidermises being formed at the interface between the liquid medium and the ambient air of the incubator. Cultivation was then continued for 1-2 weeks before reaching complete differentiation. The histological characteristics of the rebuilt three-dimensional tissue were viewed after fixing and staining with hemalum-erosine-safran (HES).

A-4-3—Evaluation of the Long Term Expansion Capacity

The keratinocytes from clonal microcultures were detached by trypsination (Gibco), and then placed in a mass culture, individually for each studied clone. These cultures were carried out on plastic surfaces on which collagen of type I was adsorbed (for example Petri, Biocoat, Becton-Dickinson plates). The culture conditions were equivalent to those used for primary clonal growth: a nutritive layer of irradiated fibroblasts, a culture medium with similar composition. After one week, the cultures reached 50%-80% confluence. The keratinocytes were then detached by trypsination, counted and then resown at a density from 2,000 to 3,000 cells/cm², under identical conditions. The cultures were then transplanted and sub-cultivated every week, until exhaustion of the expansion potential of the cells. At the end of the growth step of the primary clone and of the successive sub-cultures, cell proliferation was estimated in terms of an expansion coefficient and of the number of achieved population doublings, in order to quantify the total expansion potential of keratinocytes initially placed in culture individually. Number of population doublings=(Log N/N₀)/Log 2

N₀=Number of sown cells

N=Number of cells obtained at the end of the culture step.

A-4-4—Analysis of the Secondary Clone-Forming Capacity

The question is of estimating maintenance or loss of the potential to generate colonies from cells forming the offspring of cloned keratinocytes. To do this, the cells of primary clones were detached by trypsination (Gibco), and then sown at low density so as to obtain growth of colonies separate from each other (for example, 5 cells/cm²) under conditions similar to those described above for evaluating the long term expansion potential. After 14 days, the cultures were fixed with 70% ethanol, dried and then stained by two successive baths in eosin (RAL reagents) and in Blue RAL 555 (RAL reagents). The parameters taken into account for quantifying the clone-forming capacity of the studied cells notably were the number and size of the obtained colonies.

### B—Performance of the Means of the Invention

A parameter used for analyzing the growth potential of epithelial cells is their capacity of generating colonies in a low density culture (CFE for “colony-forming efficiency”) or clones when these are cultures sown with a single cell (CFE for “clone-forming efficiency”). It is quite obvious that the more the methods allow demonstration of high CFE values, the more they will be useful for effectively quantifying the growth potential of epithelial cells.

Table I below shows the results obtained according to two known methods for selecting and cultivating keratinocytes (they are conventionally used in the laboratory and have been described in publications), as well as the results obtained according to the method of the invention.

TABLE I

| | Phnotype | | Type of | |
|---|---|---|---|---|
| | of the | | cell | Obtained |
| Study | cells used | Clonality | material | CFE values* |
| Larderet et | ‘Side | No | Cells from | 14.5% |
| al., (2006) | population’ | | a culture | |
| | (SP) | | | |
| Rachidi et | strong α6 | No | Cells | 9.9% |
| al. (2007) | integrin/ | | extracted | |
| | weak CD71 | | from tissue | |
| Method | strong α6 | Yes | Cells | 48.4%** |
| according | integrin*** | | extracted | |
| to the | | | from tissue | |
| present | | | | |
| invention | | | | |

*CFE = % of cells capable of giving rise to a colony (low density culture situation) or to a clone (cultures with only 1 cell per well).
**Average calculated over 3 conducted experiments from independent samples.
***In a non-exclusive way.

### C—Exemplary Embodiment No. 1

The use of the model of parallel clonal microcultures for producing banks of multiple keratinocytes, each from the offspring of a single cell (FIG. 2).

C-1—Materials and Methods

Extraction of keratinocytes from an epidermis from an adult skin biopsy (mammary reduction), dissociation and suspension.

Labelling of the suspended keratinocytes with antibodies coupled with fluorochromes giving the possibility of sorting a phenotype of “stem” cells (strong expression level of integrin α6 (Itgα6) and low expression level of the receptor of transferrin (CD71): phenotype Itgα6^{strong} CD71^{weak}; selection model: Li et al., 1998).

With a cloning module by flow cytometry, automated sowing of multi-well culture plates, in an amount of one single “stem” phenotype cell per well.

After 2 weeks of culture, localization of the wells in which the cloned keratinocyte has given rise to a cell clone.

Sowing mass cultures from each clonal microculture, under conditions promoting cell multiplication.

Successive transplantations of the cultures in order to obtain the offspring of the initial cloned cells at different amplification stages (for example: a “young” or “old” culture state), and an amount of cell material stemming from the cloned cells compatible with the forming of banks of frozen cells.

Test of the maximum number of successive sub-cultures which it was possible to achieve for each culture initiated from a single keratinocyte and estimation of the total accumulated number of keratinocytes produced at the end of the long term cultures.

C-2—Results

In a cohort of 5 cell clones tested for their capability of generating a bank of keratinocytes, all of them were able to be sub-cultivated and their offspring sufficiently amplified in order to be frozen.

Two weeks after initiation of the cultures, the 5 selected clones consisted of 8.64×10⁴ to 1.11×10⁵ keratinocytes, which is equivalent to 16.40 to 16.86 successive cell generations achieved since the stage of the single cloned cell.

1 clone generated an accumulated offspring of 8.48×10¹² keratinocytes within 8 weeks of multiplication in culture (No. 1), which is equivalent to an accumulation of 42.95 average population doublings.

2 clones generated an accumulated offspring of 6.36×10¹¹ and 2.4×10¹¹ keratinocytes (No. 2 and No. 3), which is equivalent to 39.21 and 37.80 average population doublings respectively.

1 clone generated an accumulated offspring of 2.03×10¹⁰ keratinocytes within 6 weeks of multiplication in culture (No. 4), which is equivalent to an accumulation of 34.24 average population doublings.

1 clone generated an accumulated offspring of 2.15×10⁹ keratinocytes within 6 weeks of multiplication in culture (No. 5), which is equivalent to an accumulation of 31.00 average population doublings.

C-3—Conclusion

The model of parallel clonal microcultures according to the present invention represents a technology allowing standardized generation of multiple banks of keratinocytes, each from the offspring of a single cell directly isolated from a tissue sample.

It also allows estimation and comparison, at the scale of the individual cell, of the proliferation potential of distinct cell types (for example: progenitors, epidermal stem cells).

### D—Exemplary Embodiment No. 2

The use of the system of clonal microcultures for producing multiple rebuilt epidermises, each from the offspring of a single cell (FIG. 3).

D-1—Materials and Methods

Extraction of keratinocytes from an epidermis from adult skin biopsy (mammary reduction), dissociation and suspension.

Labelling the suspended keratinocytes with antibodies coupled with fluorochromes giving the possibility of sorting in flow cytometry a phenotype of “stem” cells (strong expression level of integrin α6 (Itgα6) and low expression level of the receptor of transferrin (CD71); phenotype Itgα6^{strong} CD71^{weak}; selection model: Li et al., 1998).

By means of a cloning module by flow cytometry, automated sowing of multi-well culture plates, in an amount of one single “stem” phenotype cell per well.

After 2 weeks of culture, localization of the wells in which the cloned keratinocyte has given rise to a cell clone.

The use of keratinocytes forming the cell clones separately for each of them, in order to produce rebuilt epidermises (for example: reconstruction of an epidermis on de-epidermized dead human dermis: Regnier et al., 1986).

D-2—Results

A cohort of 5 cell clones was tested for the individual capability of each clone of generating a three-dimensional rebuilt epidermis.

The experiment was conducted at a growth stage of the clones equivalent to the one described in the exemplary embodiment No. 1 (multiplication corresponding to ˜16 to 17 successive cell generations).

The 5 tested clones prove to be capable of producing an epidermis having an organization representative of that of a native epidermis.

D-3—Conclusion

The technology of parallel clonal microcultures according to the invention allows production of series of rebuilt epidermises, the particularity of which is of each being from the offspring of a single cell, while the conventionally used models for large scale test campaigns are generated from banks from a mixture of cells.

The rebuilt epidermises produced according to the method object of the present invention are generated from a cloned cell immediately after extraction from the tissue, and not after a multiplication step in culture, likely to modify the characteristics thereof.

In this exemplary embodiment, the clones were used for epidermal reconstruction at a growth stage corresponding to ˜16-17 successive cell generations. In other embodiments, epidermal reconstructions may be achieved from an earlier or more belated growth stage of the cloned cells.

The culture model according to the present invention therefore provides an original functional test allowing qualification of the organogenesis potential of cells initially placed in culture individually, immediately following selection from the tissue. It provides the possibility of evaluating the impact of a stimulus or stress at various growth stages of cells placed in culture in isolation, and then of studying the consequences thereof on the capacity of tissue reconstruction in the short or medium term after treatment.

### E—Exemplary Embodiment No. 3

The use of the model of parallel clonal microcultures for characterizing the clone-forming capacity of keratinocytes present at a tissue location of interest. In particular, the question is of estimating their capability of giving rise to a cell clone, the size of which represents an indicator of their short term multiplication potential (FIG. 4).

E-1—Materials and Methods

Extraction of keratinocytes from an epidermis from an adult skin biopsy (mammary reduction), dissociation and suspension.
Labelling the suspended keratinocytes with an antibody coupled with a fluorochrome giving the possibility of sorting a population of keratinocytes corresponding to the basal layer of the epidermis (strong expression level of integrin α6 (Itgα6): phenotype Itgα6^{strong}).
By means of the cloning module by flow cytometry, automated sowing of multi-well culture plates, in an amount of one single basal keratinocyte per well.
After 2 weeks of culture, localization of the wells in which the cloned keratinocyte has given rise to a cell clone, and then detachment and counting of the keratinocytes, individually for each clone.
Classification of the different clones according to their individual size.
   E-2—Results

The analysis of the distribution of the sizes of clones generated by keratinocytes from the basal layer of the epidermis, conducted on an accumulated cohort of ˜800 clones, reveals the functional heterogeneity of this cell compartment (FIG. 4).

At the top of this hierarchy, a minority fraction of clones is found, characterized by a significant capability of short term proliferation, the size of which may exceed 15×10⁴ keratinocytes within 2 weeks.
At the bottom of the hierarchy, on the contrary, a fraction of clones is found characterized by a very limited capability of proliferation. The size of these abortive clones does not exceed 10⁴ keratinocytes after 2 weeks of culture.
Between these extremes, the majority of the clones are found, which appear to be distributed according to a size gradient, the size value represented in majority being located around 9×10⁴ keratinocytes.
   E-3—Conclusion
The technology of parallel clonal microcultures according to the invention allows specific estimation of the individual clone-forming capability of cells from a sample of interest, in this exemplary embodiment, a preparation of basal keratinocytes of the epidermis.
In particular, the method proves to be resolvent in order to define a clonal growth profile providing a ‘functional signature’ representative of a tissue, or a sub-localization profile within a tissue, in the present case, the basal layer of the adult human interfollicular epidermis.
Further, with the system, it is possible to distinguish, within a cell sample of interest, cells having distinct potentialities depending on their short term clonal growth capacity.

The culture model according to the present invention therefore provides an original functional test allowing estimation of the clone-forming capacity of cohorts of cells individually placed in culture. A possible application is the development of quality controls achieved at the scale of the individual cell aiming at evaluating the functionality (or non-functionality) of cell samples of interest, by comparison with a validated reference. The system of clonal microcultures further provides the possibility of generating cell samples from a single cell, each individually corresponding to a specifically defined short term proliferation capacity. Another possible application consists of using the system for conducting studies aiming at analyzing the short term functional consequences of a (beneficial or toxic) treatment applied at the scale of the individual cell.

### F—Exemplary Embodiment No. 4

The use of the model of parallel clonal microcultures for characterizing the long term growth potential of keratinocytes from a sample of interest. In particular the question is of detecting the presence of keratinocytes having one of the functional properties associated with epidermal stem cells, i.e. the capability of carrying out at least 100 population doublings in culture (FIG. 5).

F-1—Materials and Methods

Extraction of keratinocytes from an epidermis from an adult skin biopsy (mammary reduction), dissociation and suspension.
Labelling of the suspended keratinocytes with an antibody coupled with a fluorochrome giving the possibility of sorting a population of keratinocytes corresponding to the basal layer of the epidermis (strong expression level of integrin α6 (Itgα6): phenotype Itgα6^{strong}).
By means of the cloning module by flow cytometry, automated sowing of multi-well culture plates, in an amount of one single basal keratinocyte per well.
After 2 weeks of culture, localization of the wells in which the cloned keratinocyte has given rise to a cell clone.
Sowing of mass cultures from a representative cohort of clonal microcultures, under conditions promoting cell multiplication.
Successive transplantations of the different cultures separately, every week, until the limit of their individual multiplication potential is reached.
Evaluating the number of successive sub-cultures which each culture of clonal origin was capable of sustaining and estimating the total accumulated number of population doublings carried out at each transplantation and at the end of the long term cultures.
   F-2—Results

The analysis of the long term growth potential of a cohort of 23 clones from basal keratinocytes reveals marked potential heterogeneity of this compartment (FIG. 5).
At the bottom of the observed potential hierarchy are found clones having a restricted growth potential, which can only be maintained in culture for 6-7 weeks and only capable of carrying out a total of 30-40 population doublings.
At the top of the potential hierarchy are found clones having a very large growth potential which may be sub-cultivated for more than 24 weeks without notable reduction of their growth capability and thus capable of exceeding the expansion level of 100 population doublings.
Between these extremes are found clones distributed according to a wide potential gradient, which may be sub-cultivated for 9-18 weeks and in majority capable of carrying out 40-80 population doublings.
   F-3—CONCLUSION
The technology or parallel clonal microcultures according to the invention allows specific estimation of the long term growth potential of the cells from a sample of interest, in this exemplary embodiment, a preparation of basal keratinocytes of the epidermis.
In particular, the method proves to be resolvent in order to distinguish clones generated from a stem cell a posteriori by their capability of carrying out accumulation of at least 100 population doublings, from clones stemming from a progenitor cell, the long term proliferation capacity of which is more limited, generally comprised between 30 and 80 population doublings.
With the system, it is possible to generate cell samples corresponding to the offspring of stem cells and of progenitor cells, the properties of which may be studied and compared to different phases of their long term proliferation.

The culture model according to the invention therefore provides an original functional test allowing qualification of the long term growth potential of cells initially placed in culture individually. For example it provides the possibility of estimating the regenerative potential of a sample, notably by evaluating the presence (or the absence) of stem cells. A possible use consists of conducting studies aiming at analyzing the long term functional consequences of a (beneficial or toxic) treatment applied at the scale of the individual cell.

### G—Exemplary Embodiment No. 5

The use of the functional test of parallel clonal microcultures for quantifying at the scale of the single isolated cell the impact of irradiation on epidermal keratinocytes of distinct phenotypes (FIG. 6).

G-1—Materials and Methods

Extraction of keratinocytes from an epidermis from an adult skin biopsy (mammary reduction), dissociation and suspension.
Labelling of the suspended keratinocytes with antibodies coupled with fluorochromes giving the possibility of sorting in flow cytometry a phenotype of “stem” cells (strong expression level of integrin α6 (strong expression level of integrin α6 (Itgα6) and weak expression level of the receptor of transferrin (CD71): phenotype (Itgα6^{strong} and CD71^{weak}) and a phenotype of “progenitor cells” (strong expression level of integrin α6 (Itgα6) and strong expression level of the receptor of transferrin (CD71): phenotype Itgα6^{strong} CD71^{strong}) (selection model: Li et al., 1998).

By means of a cloning module by flow cytometry, automated sowing of multi-well culture plates, in an amount of one single cell per well, and this for each of the two studied cell phenotypes.

Twenty hours after sowing, irradiation of each isolated cell with a single dose of 2 Gy (γ radiation).

After 2 weeks of culture, counting the wells in which a cell clone has developed, and then detachment and countings of keratinocytes, individually for each clone.

G-2—Results

G-2-1—Impact of Irradiation on the Clone-Forming Capacity of Isolated Keratinocytes (FIG. 6A)

Under a control condition (without irradiation), both tested phenotypes of keratinocytes exhibited strong capability of generating a cell clone. They were not notably distinguished on this criterion.
70.3% of individually sown keratinocytes of phenotype Itgα6^{strong} CD71^{weak} (sub-population enriched in stem cells) gave rise to a cell clone.
61.7% of keratinocytes of phenotype Itgα6^{strong} CD71^{strong} (sub-population composed of progenitors) generated a clone.

The keratinocytes of both phenotypes were on the other hand differently affected by irradiation.
The percentage of keratinocytes of phenotype Itgα6^{strong} CD71^{weak} giving rise to a clone was lowered from 70.3% (control condition) to 47.3% (irradiation of 2 Gy), which allowed maintenance of the clone-forming capacity of these cells to be estimated at 67.3%.
The clone-forming capacity of keratinocytes of phenotype Itgα6^{strong} CD71^{strong} was drastically reduced by irradiation: 61.7% of cells giving rise to a clone under a control condition versus 23.0% under an irradiation condition, which allowed maintenance of the clone-forming capacity of these cells to be estimated at only 37.3%.
   G-2-2—Impact of Irradiation on the Growth Potential of the Clones (FIG. 6B)

Under a control condition, the size of the produced clones has not proved either to be a criterion allowing clear distinction of both types of studied keratinocytes.

The keratinocytes of both phenotypes have proved to be capable of generating a large proportion of clones of large size comprising at least 5×10⁴ keratinocytes.

The analysis of the distributions of the size of the obtained clones provided parameters allowing clear distinction of the specific responses to irradiation of keratinocytes of distinct phenotypes.

Moderate reduction of the capacity of cloned keratinocytes Itgα6^{strong} CD71^{weak} of generating large size clones following irradiation (median size of the clones under a control condition: 10.1×10⁴ cells/clone; median size for the irradiated group: 6.8×10⁴ cells/clone).

Strongly marked reduction of the capacity of keratinocytes Itgα6^{strong} CD71^{strong} of producing large size clones following irradiation (median size of the clones, control group: 8.5×10⁴ cells/clone; median size for the irradiated group: 1.2×10⁴ cells/clone).

G-3—Conclusion

The model of the parallel clonal microcultures according to the invention proves to be performing for analyzing the growth capacity of keratinocytes of specific phenotypes at the scale of the individual cell. Indeed, as the values of clone-forming efficiencies obtained in this model reach 60-70% of the cloned cells, they prove to be very superior to what is generally described in conventional culture systems, concerning keratinocytes directly stemming from tissue biopsy, for which the values are of the order of 10%.

This model also proves to be performing for detecting, qualifying and quantifying a deleterious effect on the cell growth potential. The present example illustrates the capability of the system of being valued by the development of radiotoxicology tests in vitro.

### H—Exemplary Embodiment No. 6

The use of the functional test of parallel clonal microcultures for evaluating the consequences of irradiation of a single cell on the growth potential of its offspring. Comparison of the behavior of epidermal keratinocytes with distinct phenotypes (FIG. 7).

H-1—Materials and Methods

Following the procedure described in the scope of the exemplary embodiment No. 5:

Low density sowing of a portion of the keratinocytes stemming from the clones, in order to obtain individualized colonies: in this example, density of 5 keratinocytes/cm².

After 2 weeks of culture, fixation and staining of the colonies, so as to be able to carry out a microscopic and macroscopic observation.

H-2—Results

Under a control condition (without irradiation of the initial cells placed in culture individually), the groups of clones tested for their capacity of generating secondary colonies have shown very similar characteristics for this criterion.

The capacity of producing secondary colonies from the 2 series of clones, respectively stemming from keratinocytes Itgα6^{strong} CD71^{weak} [clones (1) to (5)] and from keratinocytes Itgα6^{strong} CD71^{strong} [clones (11) to (15)] has proved to be comparable.

Both of these groups comprised both cell clones abundantly giving rise to secondary colonies of large size [for example: clones (1) and (11)] and clones giving rise to not very abundant colonies and of small size [for example: clones (5) and (15)].

As regards groups of clones stemming from a cell having undergone irradiation (single dose of 2 Gy), the capacity of generating secondary colonies proves to be clearly distinct for the 2 phenotypes of the tested keratinocytes.

The group of clones stemming from keratinocytes Itgα6^{strong} CD71^{weak} [clones (6) to (10)] exhibited a capacity of producing secondary colonies equivalent to that of the non-irradiated groups.

On the other hand, the group of clones stemming from keratinoytes Itgα6^{strong} CD71^{strong} having been subject to irradiation [clones (16) to (20)] exhibited reduced secondary clone-forming capacity (losses of colonies with a diameter ≧5 mm).

H-3—Conclusion

The model of the parallel clonal microcultures according to the present invention is adapted for demonstrating, qualifying and quantifying the non-immediate consequences of irradiation carried out on individually studied keratinocytes. In the present case, the demonstrated deleterious effect was a loss of growth capacity measured on the offspring of cells placed in a clonal culture.

### I—Exemplary Embodiment No. 7

The use of the model of parallel clonal microcultures for analyzing the long term consequences of genotoxic stress applied on cells placed under a clonal condition. The question is of applying stress for a few hours after sowing the cells in separate culture wells individually, and then of initiating long term cultures after the cells have divided. The presence of abnormalities at the level of the genome is then sought at the level of the offspring of the cloned cells, after the latter has carried out a determined number of population doublings. This search is for example carried out by using a technique allowing detection of representation disequilibria of DNA sequences in the genome (deletions, amplifications) comparative genomic hybridization (CGH) (FIG. 8).

I-1—Materials and Methods

Extraction of keratinocytes from an epidermis stemming from an adult skin biopsy (mammary reduction), dissociation and suspension.
Labelling of the suspended keratinocytes with an antibody coupled with a fluorochrome giving the possibility of sorting a population of keratinocytes corresponding to the basal layer of the epidermis (strong expression level of the integrin α6 (Itgα6): phenotype Itgα6^{strong}).
By means of the cloning module by flow cytometry, automated sowing of multi-well culture plates, in an amount of one basal keratinocyte per well.
Separation of the clonal microcultures into 2 groups: 1) a group subject to a single dose of 2 Grays of gamma irradiation 19 hours after sowing; 2) a non-irradiated control group.
After 2 weeks of culture, for each of the 2 groups, localization of the wells in which the cloned keratinocyte has given rise to a cell clone.
Sowing of mass cultures from cohorts of clonal microcultures representative of each of the 2 groups, under conditions promoting cell multiplication.
Successive transplantations of the different cultures separately, every week, until the limit of their individual multiplication potential is reached.
Evaluation of the number of successive sub-cultures which each culture of clonal origin was capable of sustaining and estimation of the total accumulated number of population doublings carried out at each transplantation and at the end of the long term cultures.
From the control and irradiated groups, selection of cultures showing very significant long term proliferation capacity, notably capable of carrying out at least 150 population doublings.
For each selected candidate, preparation of genomic DNA samples corresponding to a belated long term proliferation stage, in the present case, the cultures having carried out about 150 population doublings after clonal sowings.
On the generated DNA samples, stemming from the control and irradiated groups, the search for areas of the genome having abnormalities of the deletion type and/or amplification by comparative genomic hybridization (CGH) versus reference DNA (CGH chips Constitutional Chip® 4.0, PerkinElmer, Inc.; according to a method recommended by the manufacturer).
   I-2—Results

Cytogenetic analysis by CGH chips of long term cultures of clonal origin shows that the investigated gamma ray dose of 2 Grays has the consequence that acquired chromosomal abnormalities are transmitted to the offspring, which prove to be detectable in a large number of cell divisions after applying the genotoxic stress (FIG. 8).
In the presented example, amplification of a locus with a size of 44.3 megabases located on the chromosome 10 (region 10q11.21-10q23.1) is detected at the level of the DNA of the offspring of a keratinocyte, after 150 population doublings following irradiation of the latter.
On the other hand, this same studied genomic segment at the DNA level of the offspring of 2 exemplary keratinocytes which have not been irradiated, does not exhibit this type of alteration of the genome.
   I-3—Conclusion
With the technology of clonal microcultures, it is possible to subject basal keratinocytes of the epidermis individually to a genotoxic stress, in the present case gamma irradiation, and then to evaluate the long term consequences thereof on the offspring.
In particular, the method proves to be valid for analyzing at a clonal scale, the impact of a stress on the integrity of the genome of the basal keratinocytes.
In this exemplary embodiment, the system allows detection of a genotoxic effect of gamma irradiation on keratinocytes stemming from the basal layer of the epidermis.

The culture model according to the present invention therefore provides an original system allowing toxicology tests to be conducted at the scale of the individual cell. For example, it provides the possibility of characterizing the individual sensitivity of basal keratinocytes of the epidermis to genotoxic agents. A possible use is the conducting of tests aiming at detecting the occurrence of abnormalities at the genome of the deletions and/or amplifications type, consecutively to exposure to a toxic agent, and analyzing their transmission to offspring during successive cell divisions, in particular in the long term.

### REFERENCES

Gangatirkar et al., Nat. Protoc. 2007. 2: 178-186
Li et al., Proc. Natl. Acad. Sci. USA. 1998. 95: 3902-3907
Régnier et al., Exp. Cell Res. 1986. 165: 63-72
Lorenz K et al. Cells Tissues Organs. 2008 Aug. 11.
Barrandon and Green, Proc. Natl. Acad. Sci. USA. 1987. 84: 2302-2306
Larderet et al., Stem Cells. 2006. 24: 965-974
Rachidi et al., Radiother. Oncol. 2007. 83(3):267-76.

1. A system for clonal culture of epithelial cells optimized for evaluating and exploiting specific properties of a single cell, wherein a culture support comprises at least one clonal culture sown with a single epithelial cell directly extracted from a biological sample of healthy or diseased epithelial tissue.

2. The system according to claim 1, characterized in that said culture support comprises at least two parallel clonal cultures, each of said cultures being sown with a distinct and single epithelial cell directly extracted from said biological sample.

3. The system according to claim 1 or 2, characterized in that it is a biochip.

4. A method for clonal culture of epithelial cells optimized for evaluating and exploiting specific properties of a single cell, comprising at least the steps of:
a) extracting one or more epithelial cells directly from a biological sample of healthy or diseased epithelial tissue;
b) optionally, selecting at least one population and/or sub-population of epithelial cells from the cells extracted in step a);
c) producing a clonal culture sown with a distinct and single epithelial cell directly stemming from step a) or b); and
d) qualitatively and/or quantitatively evaluating cell growth in the clonal culture of step c).

5. The method according to claim 4, characterized in that it further comprises step e) consisting of amplifying the cell population of the clonal culture of step c), or its offspring, by one or more successive sub-cultures.

6. The method according to claim 4 or 5, characterized in that it further comprises step f) consisting of using the cell population of the clonal culture of step c), or its offspring, in order to rebuild a three-dimensional tissue, so as to evaluate its tissue reconstruction potential.

7. The method according to any of the claims 4 to 6, characterized in that it further comprises step g) consisting of sub-cultivating the cell population of the clonal culture of step c), under conditions promoting cell expansion until exhaustion of the expansion potential, so as to evaluate the long term expansion potential of said cell population.

8. The method according to any of the claims 4 to 7, characterized in that it further comprises step h) consisting of evaluating the clone-forming potential of the offspring of the cell population of the clonal culture of step c), by a quantitative clonogenicity test wherein strictly clonal secondary cultures and/or low density cultures allowing growth of individualized colonies are produced.

9. The method according to any of the claims 4 to 8, characterized in that step c) comprises the production of at least two parallel clonal cultures, each of said cultures being sown with a distinct and single epithelial cell directly stemming from step a) or b).

10. The system according to any of claims 1 to 3, or the method according to any of claims 4 to 9, characterized in that said biological sample of epithelial tissue is obtained by biopsy in a mammal, preferably in humans.

11. The system according to any of claims 1 to 3 and 10, or the method according to any of claims 4 to 10, characterized in that said epithelial tissue is selected from epithelia, for example the interfollicular epidermis of adult or neonatal human skin, the cornea, the mucosas, the hair follicles.

12. The system according to any of claims 1 to 3, 10 and 11, or the method according to any of claims 4 to 11, characterized in that the epithelial cell(s) directly extracted from said biological sample is(are) healthy or diseased single cell(s) selected from progenitor cells, stem cells, keratinocytes.

13. The system according to any of claims 1 to 3, characterized in that it is obtained by applying at least steps a) to c) of the method according to claim 4.

14. A clonal cell bank obtainable at the end of step e) of the method according to claim 5.

15. A three-dimensional tissue rebuilt from a clonal culture obtainable at the end of step f) of the method according to claim 6.

16. The tissue according to claim 15, characterized in that it is selected from epithelia, the skin, the epidermis.

17. A biochip comprising at least one tissue according to claim 15 or 16.

18. The use of:
the system according to any of claims 1 to 3 and 10 to 13, or
the cell bank according to claim 14, or
the tissue according to claim 15 or 16, or
the biochip according to claim 17, or
the application of the method according to any of claims 4 to 12, for identifying and selecting agents having a biological activity of interest.

19. The use of:
the system according to any of claims 1 to 3 and 10 to 13, or
the cell bank according to claim 14, or
the tissue according to claim 15 or 16, or
the biochip according to claim 17, or
the application of the method according to any of claims 4 to 12, for in vitro diagnosis and/or in vitro prognosis in the field of cell and/or gene therapy, notably in the field of grafts.

20. The use of:
the system according to any of claims 1 to 3 and 10 to 13, or
the cell bank according to claim 14, or
the tissue according to claim 15 or 16, or
the biochip according to claim 17, or
the application of the method according to any of claims 4 to 12 for studying the behavior and/or the structural and/or functional individual properties specific to a cell, a cell sub-type, a cell sub-population, or a cell population.

21. The use of:
the system according to any of claims 1 to 3 and 10 to 13, or
the cell bank according to claim 14, or
the tissue according to claim 15 or 16, or
the biochip according to claim 17, or
the application of the method according to any of claims 4 to 12, for producing one or more tools of functional genomics.

22. The use of:
the system according to any of claims 1 to 3 and 10 to 13, or
the cell bank according to claim 14, or
the tissue according to claim 15 or 16, or
the biochip according to claim 17, or
the application of the method according to any of claims 4 to 12, in order to evaluate the efficiency or impact of treatments notably with agents having biological activity, stresses, toxic agents, genotoxic aggressions.
